(19) United States
(12) Patent Application Publication
(10) Pub. No.: US 2023/0293073 A1
(43) Pub. Date: Sep. 21, 2023

(54) ATROPHY DETECTION FOR TISSUE OCCLUDED BY IMPLANTABLE INFLATION DEVICES

(71) Applicant: Boston Scientific Scimed, Inc., Maple Grove, MN (US)

(72) Inventor: Brian P. Watschke, Minneapolis, MN (US)

(21) Appl. No.: 18/182,630

(22) Filed: Mar. 13, 2023

Related U.S. Application Data
(60) Provisional application No. 63/269,433, filed on Mar. 16, 2022.

(51) Int. Cl. A61B 5/20; A61F 2/00; A61F 2/48

(52) U.S. Cl. CPC A61B 5/20; A61F 2/004; A61F 2/484

(57) ABSTRACT

An apparatus includes a bodily implant configured to be implanted into a body of a patient. The bodily implant includes an inflatable member, a sensor, and a controller. The inflatable member is configured to be disposed within a portion of the body of the patient and place pressure on a portion of a body of th patient. The sensor is operatively coupled to the inflatable member and configured to detect a fluidic pressure within the inflatable member. The controller is configured to receive pressure data from the sensor during a transition between a deflated configuration and an inflated configuration of the inflatable member and detect, based on the received pressure data, atrophy of the portion of the body of the patient.

### CROSS-REFERENCE TO RELATED APPLICATION

This application claims priority to U.S. Provisional Patent Application No. 63/269,433, filed on Mar. 16, 2022, entitled “ATROPHY DETECTION FOR TISSUE OCCLUDED BY IMPLANTABLE INFLATION DEVICES”, the disclosure of which is incorporated by reference herein in its entirety.

### TECHNICAL FIELD

This disclosure relates generally to bodily implants, and more specifically to detection of atrophy of tissue within a body of a patient that is occluded by an inflatable implanted device.

### BACKGROUND

Implantable inflatable devices that are placed within a body of a patient can include an inflatable member that is configured to occlude a tissue of the patient for therapeutic purposes. Such devices, such as artificial urinary sphincter (AUS) device, can occlude a tissue (e.g., a urethra) of the patient for long, continuous periods of time. This occlusion can sometimes lead to atrophy of the tissue that is surrounded and/or occluded by the inflatable member tissue. In addition to symptoms and comorbidities that can be associated with such tissue atrophy, therapeutic efficacy of the device can also decrease, or be completely lost, which can lead to requiring surgical intervention to revise or replace the implantable inflatable device.

### SUMMARY

In some aspects, the techniques described herein relate to an apparatus, including: a bodily implant configured to be implanted into a body of a patient, the bodily implant including an inflatable member, a sensor, and a controller, the inflatable member being configured to be disposed within a portion of the body of the patient and to apply pressure to a portion of a body of a patient, the sensor being operatively coupled to the inflatable member and configured to detect a fluidic pressure within the inflatable member, and the controller being configured to receive pressure data from the sensor during a transition between a deflated configuration and an inflated configuration of the inflatable member and detect, based on the received pressure data, atrophy of the portion of the body of the patient.

In some aspects, the techniques described herein relate to an apparatus, wherein the inflatable member is configured to apply pressure to the portion of the body of the patient when in the inflated configuration.

In some aspects, the techniques described herein relate to an apparatus, wherein: the bodily implant includes a pump, the pump being operatively coupled to the inflatable member and configured to pump a fluid into the inflatable member to transition the inflatable member from the deflated configuration to the inflated configuration; and the detecting of the atrophy of the portion of the body of the patient is based on an amount of time the pump takes to transition the inflatable member from the deflated configuration to the inflated configuration.

In some aspects, the techniques described herein relate to an apparatus, wherein the detecting of the atrophy of the portion of the body of the patient is further based on a comparison of the amount of time the pump takes to transition the inflatable member from the deflated configuration to the inflated configuration to a baseline inflation time.

In some aspects, the techniques described herein relate to an apparatus, wherein the baseline inflation time is determined at a time the bodily implant is implanted in the body of the patient.

In some aspects, the techniques described herein relate to an apparatus, wherein: the bodily implant includes a pump, the pump being operatively coupled to the inflatable member and configured to pump a fluid out of the inflatable member to transition the inflatable member from the inflated configuration to the deflated configuration; and the detecting of the atrophy of the portion of the body of the patient is based on an amount of time the pump takes to transition the inflatable member from the inflated configuration to the deflated configuration.

In some aspects, the techniques described herein relate to an apparatus, wherein the detecting of the atrophy of the portion of the body of the patient is further based on a comparison of the amount of time the pump takes to transition the inflatable member from the inflated configuration to the deflated configuration to a baseline deflation time.

In some aspects, the techniques described herein relate to an apparatus, wherein the baseline deflation time is determined at a time the bodily implant is implanted in the body of the patient.

In some aspects, the techniques described herein relate to an apparatus, wherein: the bodily implant includes a pump, the pump being operatively coupled to the inflatable member and configured to pump a fluid into the inflatable member to transition the inflatable member from the deflated configuration to the inflated configuration; and the detecting the atrophy of the portion of the body of patient is based on a volume of fluid used to transition the inflatable member from the deflated configuration to the inflated configuration.

In some aspects, the techniques described herein relate to an apparatus, wherein the controller is configured to determine the volume of fluid based on a flow rate of the pump.

In some aspects, the techniques described herein relate to an apparatus, wherein the detecting of the atrophy of the portion of the body of the patient is further based on a comparison of the volume of fluid used to transition the inflatable member from the deflated configuration to the inflated configuration to a baseline volume of fluid used to transition the inflatable member from the deflated configuration to the inflated configuration that is determined at a time the bodily implant is implanted in the body of the patient.

In some aspects, the techniques described herein relate to an apparatus, wherein: the bodily implant includes a pump, the pump being operatively coupled to the inflatable member and configured to pump a fluid out of the inflatable member to transition the inflatable member from the inflated configuration to the deflated configuration; and the detecting the atrophy of the portion of the body of patient is based on a volume of fluid used to transition the inflatable member from the inflated configuration to the deflated configuration.

In some aspects, the techniques described herein relate to an apparatus, wherein the controller is configured to determine the volume of fluid based on a flow rate of the pump.

In some aspects, the techniques described herein relate to an apparatus, wherein the detecting of the atrophy of the portion of the body of the patient is further based on a comparison of the volume of fluid used to transition the inflatable member from the inflated configuration to the deflated configuration to a baseline volume of fluid used to transition the inflatable member from the inflated configuration to the deflated configuration that is determined at a time the bodily implant is implanted in the body of the patient.

In some aspects, the techniques described herein relate to an apparatus, where the inflatable member is an inflatable cuff.

In some aspects, the techniques described herein relate to an apparatus, wherein the inflatable cuff is an artificial urinary sphincter.

In some aspects, the techniques described herein relate to an apparatus, wherein: the portion of the body of the patient is a urethra of the patient; and the pressure applied by the inflatable member occludes a lumen of the urethra.

In some aspects, the techniques described herein relate to a method including: transitioning an inflatable member of bodily implant implanted into a body of a patient between a deflated configuration and an inflated configuration, the inflatable member being configured to be disposed within a portion of the body of the patient and to apply pressure to a portion of a body of a patient; determining, during the transition, pressure data corresponding with fluidic pressure fluidic pressure within the inflatable member, and detecting, based on the pressure data, atrophy of the portion of the body of the patient.

In some aspects, the techniques described herein relate to a method, wherein: the transition is from the deflated configuration to the inflated configuration; and the detecting of the atrophy of the portion of the body of the patient is based on one of: an amount of time to transition the inflatable member from the deflated configuration to the inflated configuration; and a volume of fluid used to transition the inflatable member from the deflated configuration to the inflated configuration.

In some aspects, the techniques described herein relate to a method, wherein: the transition is from the inflated configuration to the deflated configuration; and the detecting of the atrophy of the portion of the body of the patient is based on one of: an amount of time to transition the inflatable member from the inflated configuration to the deflated configuration; and a volume of fluid used to transition the inflatable member from the inflated configuration to the deflated configuration.

### BRIEF DESCRIPTION OF THE DRAWINGS

FIG. 1 is a schematic illustration of an implantable inflation device according to an aspect.

FIGS. 2A and 2B are schematic illustrations of implantable inflation devices according to additional aspects.

FIG. 3 is a graph illustrating a comparison of pressure data during inflation of an implantable device simulating tissue atrophy according to an aspect.

FIG. 4 is a graph illustrating an approach for detecting tissue atrophy using the data of FIG. 3 after curve smoothing according to an aspect.

FIG. 5 illustrates an example of an electronic pump assembly according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or where the methods disclosed for operating the medical device by the present disclosure are implemented.

The implantable inflation devices (bodily implants) and related methods described herein can be used to detect atrophy of a tissue of a patient that can be caused by such inflation devices when used for therapeutic purpose. Such tissue atrophy can be a result of occlusion pressures placed on the tissue by an inflatable member of a bodily implant. For instance, higher occlusion pressures may lead to an increased risk of such tissue atrophy. Because current embodiments of bodily implant devices do not have the ability for early detection and identification of tissue atrophy, the occurrence of such atrophy is manifested through other outward symptoms, such as pain, tissue trauma, and/or loss of therapeutic efficacy. Once such outward symptoms present, the patient may require surgical intervention to resolve effects of the tissue atrophy, which may then not be reversible.

The bodily implants and related methods as described herein can provide for early detection of an occurrence of tissue atrophy, which can facilitate early clinical intervention, such as modification of therapy, to prevent further atrophy, or potentially reverse the detected tissue atrophy. For instance, the devices descried herein can monitor and adjust occlusion pressures of an inflatable member. In such embodiments, such monitoring and adjustment of a patient's therapy can also be used to detect and prevent potential urethral atrophy without the need for surgical intervention, or without the need for such atrophy to present through outward symptoms. In some embodiments, the inflatable member can be an inflatable cuff used as an artificial urinary sphincter (AUS) to occlude a urethra of a patient for treatment of urinary incontinence.

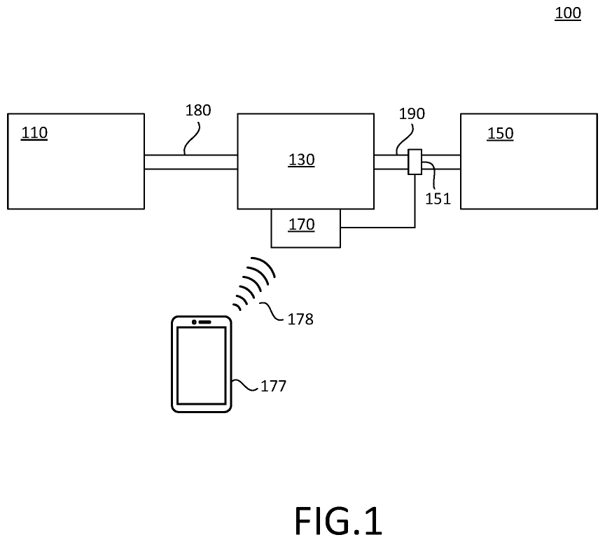

FIG. 1 is a schematic illustration of an implantable inflation device 100, or bodily implant, according to an aspect. The device 100, as well as other example devices described herein, is configured to detect potential atrophy of a tissue of a patient caused by occlusion pressure applied by an inflatable member the device 100.

The device 100 includes a fluid reservoir 110, a pump assembly 130, and an inflatable member 150. In some embodiments, the fluid reservoir 110 can be a pressure regulating balloon (PRB). As shown in FIG. 1, the fluid reservoir 110 is operatively or fluidically coupled to the pump assembly 130 via a connection member 180. The connection member 180 may be a tubular member such as a kink resistant tubing (KRT). In other embodiments, the fluid reservoir 110 is operatively or fluidically coupled to the pump assembly 130 via a different mechanism. Similarly, the inflatable member 150 is operatively or fluidically coupled to the pump assembly 130 via a connection member 190. The connection member 190 may be a tubular member such as a kink resistant tubing (KRT). In other embodiments, the inflatable member 150 is operatively or fluidically coupled to the pump assembly 130 via a different mechanism.

The implantable inflation device 100 may be configured to be implanted into a body of a patient or user. For example, in some embodiments, the implantable inflation device 100 is a AUS. In such embodiments, the inflatable member 150 may be implanted so as to surround a urethra of the patient or user. The fluid reservoir 110 may be implanted in the abdomen or pelvic cavity of the user (e.g., the fluid reservoir 110 may be implanted in the lower portion of the user's abdominal cavity or the upper portion of the user's pelvic cavity), and the pump assembly 130, and an associated control module 170, may be implanted into a portion of the body of the user, such as an abdomen of the user In other embodiments, the implantable inflation device 100 is implanted into a different portion of the body of the patient and/or is implanted for a different purpose.

The pump assembly 130 may include a pump, or more than one pump, that is configured pump fluid from the reservoir 110 into the inflatable member 150 during an inflation cycle. In some examples, the pump or pumps may be mechanically and/or programmatically controlled by the control module 170. In some embodiments, the fluid reservoir 110 can be a PRB and the pump assembly 130 can include a valve that regulates flow of inflation fluid from the fluid reservoir 110 to the inflatable member 150.

The inflatable member 150 may be capable of expanding upon the injection of fluid into a cavity of the inflatable member 150. In some embodiments, the inflatable member 150 may include a cylindrical cuff, or AUS, that can be implanted with a body of a patient around a junction between the patient's bladder and ureter, e.g., such a patient's urethra, as a treatment for incontinence. For instance, the patient can inflate and deflate the inflatable member 150 (AUS) to control the flow of urine. That is, when the inflatable member 150 is inflated, the urethra can be occluded and a flow of urine inhibited, while deflating the inflatable member 150 allows for the flow of urine and the patient to void their bladder through control of the inflatable member 150. In AUS implementations, the cylindrical cuff can include a rigid external backing, such that the cuff only expands inwardly when inflated.

The fluid reservoir 110 may include a container having an internal cavity or chamber configured to hold or house fluid that is used to inflate the inflatable member 150. The volumetric capacity of the fluid reservoir 110 may vary. In some examples, the volumetric capacity of the fluid reservoir 110 may be 3 to 150 cubic centimeters. In some examples, the fluid reservoir 110 is constructed from the same material as the inflatable member 150. e.g., silicone. In other examples, the fluid reservoir 110 is constructed from a different material than the inflatable member 150. In some examples, the fluid reservoir 110 can be sized to contain a larger volume of fluid than the inflatable member 150.

The device 100 also includes a pressure sensor 151 that can be disposed in a fluidic pathway of the connection member 190, or can be otherwise operatively coupled with the inflatable member 150 or the connection member 190 to sense a fluidic pressure in the inflatable member 150. As shown in FIG. 1, in this example, the pressure sensor 151 is operatively coupled with the control module 170, and the control module 170 can be configured to receive pressure data from the pressure sensor 151 to use in implementing the approaches described herein. That is, the control module 170 can be configured to receive and monitor occlusion pressures applied by the inflatable member 150, determine an amount of time the pump assembly 130 takes to inflate the inflatable member 150, and/or determine a volume of fluid used to achieve a target occlusion pressure. The control module 170 can also be configured to detect potential tissue atrophy, e.g., of the patient's urethra, based on comparisons of inflation times and/or inflation volumes with baseline values. In some embodiments, baseline values can be determined at a time when the implant is implanted in a body of a patient, can be values determined by a clinician when modifying treatment therapy, etc.

In some embodiments, the control module 170 can be configured to receive and monitor occlusion pressures applied by the inflatable member 150 during a transition from the inflated configuration the deflated configuration, determine an amount of time the pump assembly 130 takes to deflate the inflatable member 150, and/or determine a volume of fluid used to achieve a target deflation pressure. The control module 170 can also be configured to detect potential tissue atrophy, e.g., of the patient's urethra, based on comparisons of deflation times and/or deflation volumes with baseline values. In some embodiments, baseline values can be determined at a time when the implant is implanted in a body of a patient, can be values determined by a clinician when modifying treatment therapy, etc.

In the illustrated embodiment of FIG. 1, the control module 170 is also operatively coupled to the pump assembly 130. In some embodiments, the pump assembly 130, the control module 170 and/or the pressure sensor 151 can be integrated in a single component or module, and included in a common housing that can be implanted in a body of a patient. In some embodiments, the control module 170 is configured to activate and deactivate the pump or pumps of the pump assembly 130, such as in response to an external controller 177, which may communicate with the control module 170 via a wireless link 178, which can be a bi-directional radio link. Accordingly, the control module 170 can be configured to activate or deactivate the pump or pumps at patient or user's request, e.g., in response to a signal from the external controller 177, in order to control the inflation pressure or the state (inflated state or deflated state) of the inflatable member 150, such as to inflate the inflatable member 150 to a target occlusion pressure. In some embodiments, the external controller 177 can be implemented using a smartphone application.

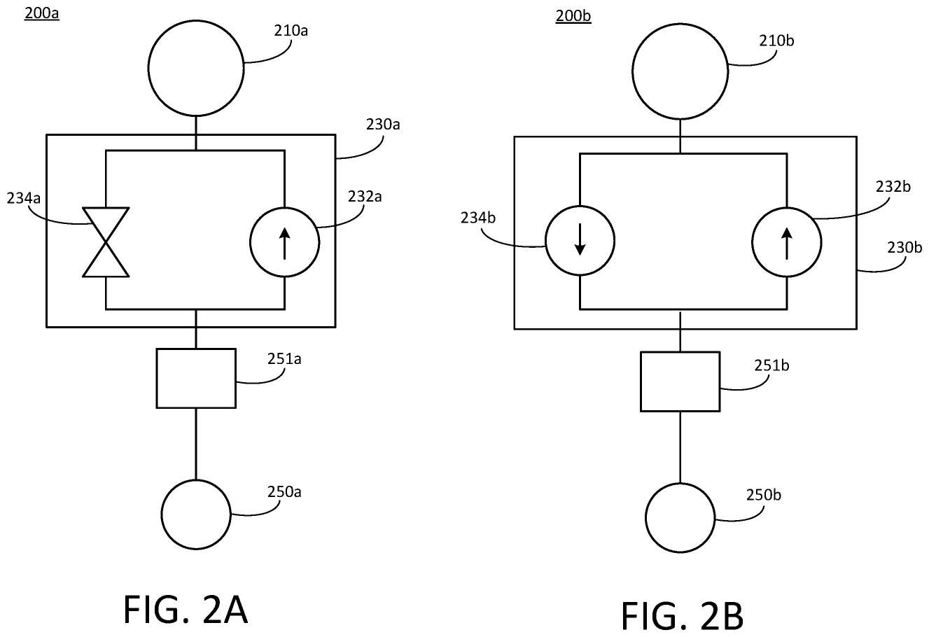

FIGS. 2A and 2B are schematic illustrations of implantable inflation devices according to additional aspects, e.g., a device 200a and a device 200b, respectively. The devices 200a and 200b are provided by way of example and for purpose of illustration as embodiments of bodily implants in which the approaches described herein can be implemented.

Referring to FIG. 2A, the implantable inflation device 200a includes a pressure regulating balloon 210a, a pump assembly 230a, an inflatable AUS 250a and a pressure sensor 251a, which can be fluidically coupled with each other as shown. In this example, the pressure regulating balloon 210a acts as a fluid reservoir for the device 200a and provides fluidic pressure for inflating the inflatable AUS 250a, e.g., to occlude a patient's urethra. The pump assembly 230a includes a pump 232a and a valve 234a. When the inflatable AUS 250a is inflated, e.g., with an inflation fluid such as saline, the pump 232a can be used to deflate the inflatable AUS 250a, so that the patient can void their bladder. After the patient has voided their bladder, the patient can then indicate that the inflatable AUS 250a should be reinflated, such as using an external controller.

In the device 210a, the pressure sensor 251a can be configured to measure a fluidic pressure in the inflatable AUS 250a in order to ensure that a target occlusion pressure is applied when the inflatable AUS 250a is inflated. For instance, the pressure sensor 251a can serve as a feedback mechanism to a control module control. The control module can then control both the valve 234a and the pump 232a, e.g., in order to maintain the target occlusion pressure in the inflatable AUS 250a, or to control deflation of the inflatable AUS 250a when request by the patient.

Referring to FIG. 2B, the device 200b includes a non-pressurized fluid reservoir 210b, a pump assembly 230b, an inflatable AUS 250b and a pressure sensor 251b, which can be fluidically coupled with each other as shown. In this example, as compared with the device 210a of FIG. 2A, the pump assembly 230b includes a pump 232b that is used to deflate the 205b, and a pump 234b that is used to inflate the inflatable AUS 250b. Similar to the device 200a, the pressure sensor 251b of the inflatable AUS 250b can be configured to measures a fluidic pressure in the inflatable AUS 250b in order to ensure that a target occlusion pressure is applied. For instance, the pressure sensor 251b can serve as a feedback mechanism to a control module control, which can control both the pump 232b and the pump 234b, in order to maintain the target occlusion pressure in the inflatable AUS 250b, or to control deflation of the inflatable AUS 250b.

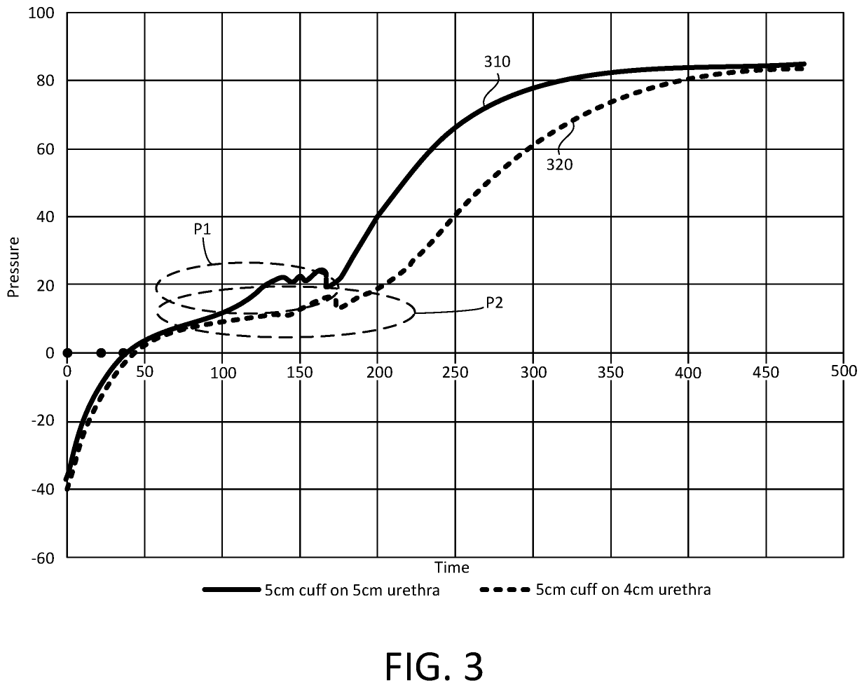

FIG. 3 is a graph illustrating a comparison of pressure data during inflation of an implantable device that simulates tissue atrophy compared to a pre-atrophy (baseline) according to an aspect. The data shown in FIG. 3 illustrates fluidic pressure (cmH2O) within a 5 centimeter (cm) internal diameter AUS over time as it is inflated, e.g., using a pump or PRB to inject inflation fluid into the AUS. In this example, trace 310 illustrates occlusion of a 5 cm urethra (e.g., occlusion of a lumen with the urethra), which can represent a baseline data urethral diameter. Also in FIG. 3, trace 320 illustrates data for occlusion of a 4 cm urethra, which can represent atrophy of the 5 cm baseline. The initial negative pressures shown by both sets of data In FIG. 3 are due to a deflation pump evacuating the AUS, such that a vacuum is created in AUS due to the removal of inflation fluid.

As also shown by each set of data in FIG. 3, as inflation begins, the fluidic pressure in the AUS rises rapidly to a first plateau (P1 for the trace 310 and P2 for the trace 320) as inflation fluid is transferred, or pumped into the AUS, e.g., from a corresponding fluid reservoir, either via a pump or as a result of a pressure differential of a PRB. These plateaus, P1 and P2, are at relatively low fluidic pressures, as compared to a target occlusion pressure. That is, the respective fluidic pressures during the P1 and P2 plateaus are due to the AUS being filled with inflation fluid, but not yet applying pressure to the respective urethra. Once the AUS is filled enough to start applying pressure to the respective urethras, the pressures within the AUS, as shown for both data sets, again begins to rise until a second plateau is reached, where the second plateau corresponds with a final desired occlusion pressure, or target occlusion pressure that is placed on the urethra.

For each of the traces in FIG. 3, the respective amount of time that is spent in the first plateaus, P1 and P2, corresponds to an amount of time it takes the AUS to fill to the point it begins to apply pressure to the urethra, e.g., after the initial period where transitions from an internal negative fluidic pressure (vacuum) to a positive fluidic pressure as the AUS begins to fill with inflation fluid.

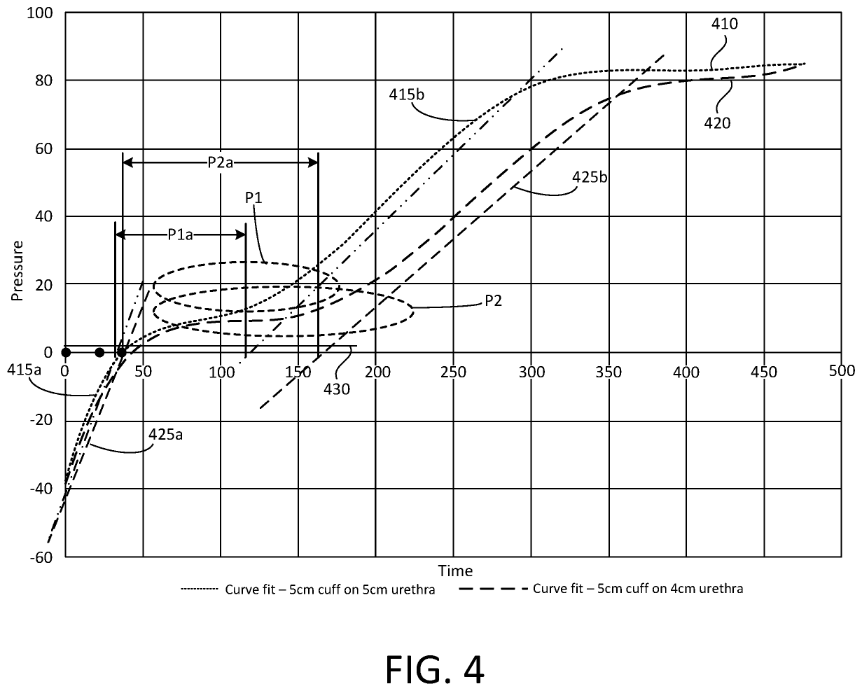

As can be seen by a comparison of P1 and P2 in the data of FIG. 3, the greater the gap between the inner, uninflated diameter of the AUS and the urethra (no gap for the trace 310 and a 1 cm diameter differential for the trace 320), the longer the amount of time that is spent in a corresponding plateau region. Accordingly, configuring a controller to measure respective amounts of time for P1 and P2 (such as illustrated by FIG. 4), and determine a difference between those times can be used as an approach to indicate if any changes, such as tissue atrophy, are occurring over time and use of the AUS. For example, if at a time of implant (baseline) a time spent in the plateau region, e.g., P1, for an AUS is 15 seconds, and 3 months after implanting the AUS, a time spent in the plateau region, e.g., P1, for an AUS is consistently 25 seconds, this difference could indicate that atrophy of the occluded tissue (e.g., the patient's urethra) has occurred.

In some bodily implant embodiments, referring to the example of FIG. 3, an amount of atrophy can be determined based on a difference between a baseline volume of fluid used in P1 to achieve a target occlusion pressure and a volume of fluid used in P2 to achieve a target occlusion pressure. Such an approach would be effective for determining an amount of occlusion based on this volumetric difference, as AUS cuffs are generally implemented as cylinders of a known outer diameter, and include a rigid backing, such that a volume of fluid used to inflate the AUS can be used determine its inner diameter. For instance, the inflatable portion of the AUS can only expand inward due to restriction of outward expansion by the rigid backing. Accordingly, a difference between two such inner diameters, e.g., baseline and after detection atrophy, would correspond to an amount of urethral atrophy, which could be determined by a difference between simple, respective volume calculations.

In the data of the FIG. 3, the variations observed in the fluidic pressure data, e.g., during P1 and P2, are due, at least in part, to pillows that are formed as the AUS inflates, as well as interaction between the AUS and the surrounded urethral tissue, e.g., as the AUS comes into contact and then releases and or/shifts on the urethra until complete circumferential coaptation occurs. At this point, the respective fluidic pressures in the AUS rise until the final or target occlusion pressure is reached. Even though the data in FIG. 3 has noise and environmental artifacts, the data demonstrates that the plateau dwell time for P2 is longer than the plateau dwell time for P1, which can be indicative of atrophy, e.g., a 4 cm atrophied urethral diameter as opposed to a 5 cm baseline urethral diameter. In the implementations described herein, a control module can be configured to apply curve smoothing to pressure data captured by a pressure sensor, e.g., the data in FIG. 3, where such smoothed or curve fit pressure data is shown in FIG. 4. That smoothed data can then be used to identify the potential occurrence of tissue atrophy.

As indicated above, FIG. 4 is a graph illustrating an approach for detecting tissue atrophy using the data of FIG. 3 after curve smoothing according to an aspect. The trace 400 and trace 420 are curve fit, or smoothed pressure data corresponding, respectively, with the trace 310 and the trace 320 of FIG. 3. In FIG. 4, line 415a and line 415b are fit lines to the slopes of fluidic pressures within the AUS versus time before and after the initial plateau region P1, respectively, for the trace 410. Similarly, line 425a and line 425b are fit lines to the slopes of fluidic pressures within the AUS versus time before and after the initial plateau region P2, respectively, for the trace 420. The line 430 is a datum used to approximate, or represent the plateaus P1 and P2, and also serve as an intersection point for the dashed slope lines. The distance between these two intersections, shown as P1a and P2a, can be used to compare how a corresponding tissue, e.g., a patient's urethra, is responding to an applied therapeutic occlusion pressure over time, and to determine if atrophy of that tissue is occurring.

As mentioned above, the times P1a and P2a in FIG. 4 correspond to the respective times spent in the first plateau regions, e.g., a low-pressure state as the AUS is filling to the point of completely contacting the surrounded tissue. To determine whether atrophy is occurring, a threshold change value can be used. For instance, an increase of time spent in the first plateau region of 15% could be used. In this example, an increase of P2a over P1a of less than 15% could be considered to indicate that atrophy has not occurred, while an increase of P2a over P1a of 15% or more could be considered to indicate that atrophy has occurred. In example embodiments, a control module of a corresponding bodily implant can be configured to notify the patient, or a physician of an indication of potential atrophy. Such notifications could be provided in various ways, such as by a remote communication to the physician, e.g., over data network accessible to the controller, during physician interrogation of the device. Alternatively, the control module could notify the patient to contact their physician, e.g., via an external controller for the bodily implant, as another example. Further, even if the atrophy indication threshold is not met, a trend can be observed and used to predict potential future issues.

Obviously, the amount of time spent in this plateau regions discussed above, depends on how fast the AUS is being filled. The initial pressure slopes shown in FIG. 4 (line 415a and line 425a) can be used to determine a corresponding fill rate, as it takes very little fluid to move from the AUS from a vacuum state to the first plateau. By determining the respective fill rates for the AUS, any corresponding atrophy determinations can be adjusted for such differences in fill rate, e.g., when comparing respective amounts of time spent in the plateau regions (e.g., P1a and P2a). This adjustment can be important when using electronic pumps, as no two pumps will pump at the exact same speed. Additionally, over time and use, the efficiency or performance of a given pump may change.

In some embodiments, a determination, or detection of atrophy can be made based on transitioning an inflatable member, such as those described herein, from an inflated configuration to a deflated configuration. In such embodiments, the pressure data used could be pressure data that is substantially the reverse, or inverse of the pressure data shown in FIGS. 3 and 4.

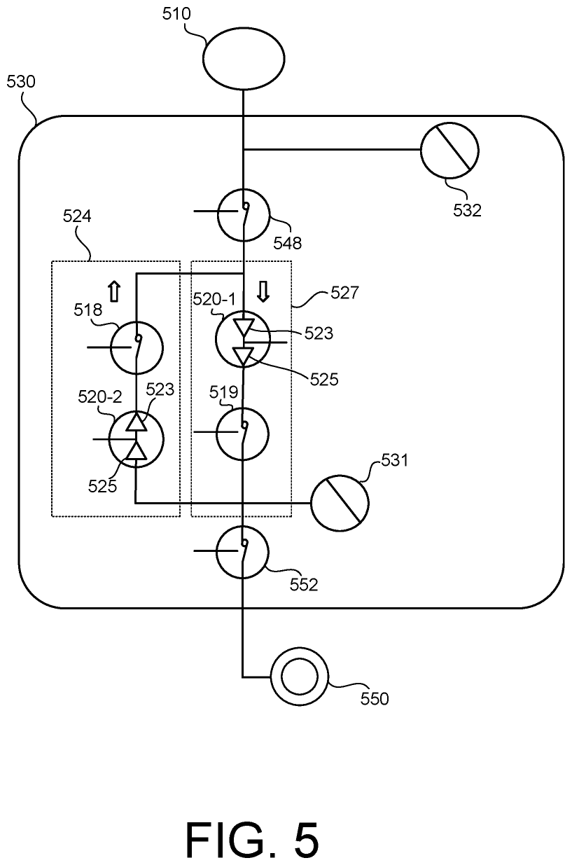

FIG. 5 illustrates an example of a portion of an electronic pump assembly 530 according to an aspect. The electronic pump assembly 530, or portions thereof, may be an example of the pump assembly 130 of FIG. 1, and/or the respective pump assemblies 230a and 230b of the devices of FIGS. 2A and 2B, and may include any of the details discussed with reference to the inflatable devices, or bodily implants described herein.

The electronic pump assembly 530 is configured to transfer fluid between the fluid reservoir 510 and the inflatable member 550 (e.g., an inflatable AUS cuff). The electronic pump assembly 530 may transfer fluid between the fluid reservoir 510 and the inflatable member 550 via one more pumps without the user manually operating a pump (e.g., squeezing and releasing a pump bu lb).

For instance, the electronic pump assembly 530 includes a pump 520-1 disposed within a fluid passageway 527 (e.g., a fill passageway), and an active valve 518 disposed within a fluid passageway 524 (e.g., an empty passageway). The pump 520-1 may be an electromagnetic pump or a Piezoelectric pump. The pump 520-1 may include a passive check valve 523 and a passive check valve 525. The fluid passageway 527 may be a fluid branch that is separate (and parallel) to the fluid passageway 524. The fluid passageway 527 is the passageway that transfers fluid from the fluid reservoir 510 to the inflatable member 550. The fluid passageway 524 is the passageway that transfers fluid from the inflatable member 550 to the fluid reservoir 510. The pump 520-1 is disposed in parallel with the active valve 518.

In some examples, the electronic pump assembly 530 may include an active valve 519 in series with the pump 520-1 (e.g., the pump 520-1 and the active valve 519 are disposed within the fluid passageway 527). In some examples, the electronic pump assembly 530 may include a pump 520-2 in series with the active valve 518 (e.g., the pump 520-2 and the active valve 518 are disposed in the fluid passageway 524). The pump 520-2 may be an electromagnetic pump or a Piezoelectric pump. The pump 520-2 may include a passive check valve 523 and a passive check valve 525. In some examples, the electronic pump assembly 530 includes an active valve 548 that is fluidly connected to the fluid reservoir 510. The active valve 548 may be in series with either the active valve 518 (and the pump 520-2) or the pump 520-1 (and the active valve 519). In some examples, the electronic pump assembly 530 includes an active valve 552 that is fluidly connected to the inflatable member 550. The active valve 552 may be in series with either the active valve 519 (and the pump 520-1) or the pump 520-2 (and the active valve 518).

The active valve 548, the pump 520-1, the active valve 518, the active valve 552, the active valve 518, and the pump 520-2 may be electronically controlled by a controller and/or driver (e.g., the control module 170 of FIG. 1, and/or control modules implemented in conjunction with the devices of FIGS. 2A and 2B). The pump 520-1 and the pump 520-2 may be unidirectional or bidirectional. With respect to the fluid passageway 527, in some examples, the pump 520-1 and the active valve 519 may swap positions (e.g., where the active valve 519 is in series between the active valve 548 and the pump 520-1). With respect to the fluid passageway 524, in some examples, the active valve 518 and the pump 520-2 may swap positions (e.g., where the pump 520-1 is in series with and between the active valve 518 and the active valve 548).

In some examples, one or more additional active valves and/or one or more additional pumps are disposed in series within the fluid passageway 527. In some examples, one or more additional active valves and/or one or more additional pumps are disposed in series within the fluid passageway 524. In some examples, the electronic pump assembly 530 may include one or more additional (and parallel) fluid passageways, where each additional (and parallel) fluid passageway may include one or more active valves and one or more pumps.

In some examples, such as those described herein, the electronic pump assembly 530 may include a pressure sensor 531 and a pressure sensor 532. The pressure sensor 531 and the pressure sensor 532 are connected to a controller e.g., the control module 170 of FIG. 1, and/or control modules implemented in conjunction with the devices of FIGS. 2A and 2B), where the controller receives the measured pressures, respectively, from the pressure sensor 531 and the pressure sensor 532 and can use those pressure readings, along with related fill rate information, to detect tissue atrophy in a patient as described herein.

For instance, the pressure sensor 531 is configured to measure the fluidic pressure in the inflatable member 550. The controller may receive the measured pressure from the pressure sensor 531 and, in response, automatically control the active valves and/or the pump to regulate the pressure, and/or provide notifications regarding detection of potential tissue atrophy. In some examples, the pressure sensor 532 is configured to measure the pressure in the fluid reservoir 510. In some examples, the pressure sensor 532 may detect intra-abdominal pressure (which can increase during activities such as exercise, and the controller can control the active valves and pump to minimize or prevent accidental inflations. In some examples, the electronic pump assembly 530 may include one or more pressure sensors at other locations within the electronic pump assembly 530. For example, a pressure sensor may be disposed between the active valve 548 and the pump 520-1. In some examples, a pressure sensor may be disposed between the pump 520-1 and the active valve 519. In some examples, a pressure sensor may be disposed between the active valve 548 and the active valve 518. In some examples, a pressure sensor may be disposed between the active valve 518 and the pump 520-2. In some examples, a pressure sensor may be placed between the inflatable member 550 and the active valve 552.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An apparatus, comprising:
a bodily implant configured to be implanted into a body of a patient, the bodily implant including an inflatable member, a sensor, and a controller,
the inflatable member being configured to be disposed within a portion of the body of the patient and to apply pressure to a portion of a body of a patient,
the sensor being operatively coupled to the inflatable member and configured to detect a fluidic pressure within the inflatable member, and
the controller being configured to receive pressure data from the sensor during a transition between a deflated configuration and an inflated configuration of the inflatable member and detect, based on the received pressure data, atrophy of the portion of the body of the patient.

2. The apparatus of claim 1, wherein the inflatable member is configured to apply pressure to the portion of the body of the patient when in the inflated configuration.

3. The apparatus of claim 1, wherein:
the bodily implant includes a pump, the pump being operatively coupled to the inflatable member and configured to pump a fluid into the inflatable member to transition the inflatable member from the deflated configuration to the inflated configuration; and
the detecting of the atrophy of the portion of the body of the patient is based on an amount of time the pump takes to transition the inflatable member from the deflated configuration to the inflated configuration.

4. The apparatus of claim 3, wherein the detecting of the atrophy of the portion of the body of the patient is further based on a comparison of the amount of time the pump takes to transition the inflatable member from the deflated configuration to the inflated configuration to a baseline inflation time.

5. The apparatus of claim 4, wherein the baseline inflation time is determined at a time the bodily implant is implanted in the body of the patient.

6. The apparatus of claim 1, wherein:
the bodily implant includes a pump, the pump being operatively coupled to the inflatable member and configured to pump a fluid out of the inflatable member to transition the inflatable member from the inflated configuration to the deflated configuration; and
the detecting of the atrophy of the portion of the body of the patient is based on an amount of time the pump takes to transition the inflatable member from the inflated configuration to the deflated configuration.

7. The apparatus of claim 6, wherein the detecting of the atrophy of the portion of the body of the patient is further based on a comparison of the amount of time the pump takes to transition the inflatable member from the inflated configuration to the deflated configuration to a baseline deflation time.

8. The apparatus of claim 7, wherein the baseline deflation time is determined at a time the bodily implant is implanted in the body of the patient.

9. The apparatus of claim 1, wherein:
the bodily implant includes a pump, the pump being operatively coupled to the inflatable member and configured to pump a fluid into the inflatable member to transition the inflatable member from the deflated configuration to the inflated configuration; and
the detecting the atrophy of the portion of the body of patient is based on a volume of fluid used to transition the inflatable member from the deflated configuration to the inflated configuration.

10. The apparatus of claim 9, wherein the controller is configured to determine the volume of fluid based on a flow rate of the pump.

11. The apparatus of claim 10, wherein the detecting of the atrophy of the portion of the body of the patient is further based on a comparison of the volume of fluid used to transition the inflatable member from the deflated configuration to the inflated configuration to a baseline volume of fluid used to transition the inflatable member from the deflated configuration to the inflated configuration that is determined at a time the bodily implant is implanted in the body of the patient.

12. The apparatus of claim 1, wherein:
the bodily implant includes a pump, the pump being operatively coupled to the inflatable member and configured to pump a fluid out of the inflatable member to transition the inflatable member from the inflated configuration to the deflated configuration; and
the detecting the atrophy of the portion of the body of patient is based on a volume of fluid used to transition the inflatable member from the inflated configuration to the deflated configuration.

13. The apparatus of claim 12, wherein the controller is configured to determine the volume of fluid based on a flow rate of the pump.

14. The apparatus of claim 13, wherein the detecting of the atrophy of the portion of the body of the patient is further based on a comparison of the volume of fluid used to transition the inflatable member from the inflated configuration to the deflated configuration to a baseline volume of fluid used to transition the inflatable member from the inflated configuration to the deflated configuration that is determined at a time the bodily implant is implanted in the body of the patient.

15. The apparatus of claim 1, where the inflatable member is an inflatable cuff.

16. The apparatus of claim 15, wherein the inflatable cuff is an artificial urinary sphincter.

17. The apparatus of claim 1, wherein:
the portion of the body of the patient is a urethra of the patient; and
the pressure applied by the inflatable member occludes a lumen of the urethra.

18. A method comprising:
transitioning an inflatable member of bodily implant implanted into a body of a patient between a deflated configuration and an inflated configuration, the inflatable member being configured to be disposed within a portion of the body of the patient and to apply pressure to a portion of a body of a patient;
determining, during the transition, pressure data corresponding with fluidic pressure fluidic pressure within the inflatable member, and
detecting, based on the pressure data, atrophy of the portion of the body of the patient.

19. The method of claim 18, wherein:
the transition is from the deflated configuration to the inflated configuration; and
the detecting of the atrophy of the portion of the body of the patient is based on one of:
an amount of time to transition the inflatable member from the deflated configuration to the inflated configuration; and
a volume of fluid used to transition the inflatable member from the deflated configuration to the inflated configuration.

20. The method of claim 18, wherein:
the transition is from the inflated configuration to the deflated configuration; and
the detecting of the atrophy of the portion of the body of the patient is based on one of:
an amount of time to transition the inflatable member from the inflated configuration to the deflated configuration; and
a volume of fluid used to transition the inflatable member from the inflated configuration to the deflated configuration.
